(19) United States
(12) Patent Application Publication
(10) Pub. No.: US 2023/0149571 A1
(43) Pub. Date: May 18, 2023

(54) Apparatus and Methods for Inactivating Bacteria on Surfaces and Mammalian Tissue

(71) Applicant: Denny D. Beasley, LaGrange Park, IL (US)

(72) Inventor: Denny D. Beasley, LaGrange Park, IL (US)

(21) Appl. No.: 17/916,583

(22) PCT Filed: Apr. 5, 2021
(86) PCT No.: PCT/US21/25763

§ 371 Date: Oct. 2, 2022

Related U.S. Application Data
(60) Provisional application No. 63/005,139, filed on Apr. 3, 2020.

(51) Int. Cl. A61L 2/00

(52) U.S. Cl. CPC A61L 2/0047; A61L 2/0094; A61L 2/0058; A61L 2202/11; A61L 2202/15

(57) ABSTRACT

A device to provide sterilized surfaces and skin with the application of multiple bactericidal agents in combinations accentuating each agent's efficacy. UV, IR, and potentially others, in conjunction with minimal Ozone levels conditioned to rapidly move through the air surface/boundary layer for effective and timely activation of oxidizing effects on bacterial agents and secondarily through surface layer absorption for continued bacterial inactivation/oxidation after the stenlization event has occurred.

### CROSS REFERENCE TO RELATED APPLICATION

This application claims the benefit of the filing date of U.S. Provisional Patent Application Ser. No. 63/005,139, filed Apr. 3, 2020, which is hereby incorporated by reference in its entirety.

### BACKGROUND

Bacterial elimination has become an important endeavor in protecting and preventing diseases. Bactericides, such as alcohol and chlorine, and photonic energies, such as ultraviolet (“UV”) light, have been employed to kill or disable bacteria, but these bactericides have side effects that compromise the safety and health of the treatment subject. Single-element bactericidal treatments are limited by a single modality of inactivation. For example, UV having a 254 nm wavelength acts at the chromosomal level by interrupting DNA structure of bacteria, whereas bactericides in the form of chemical agents (e.g., chlorine and Ozone) breakdown the bacteria cell's membrane lipid layers by oxidation and rupture the bacteria cell walls. In operation, the sterilization effects of these bactericide and photonic energy sources, when applied to mammalian surfaces, should have concentrations well below maximum efficacy to be safe for human contact.

### SUMMARY

The present disclosure provides apparatus, methods of use, and methods of treatment to beneficially inactivate bacteria on a treatment surface, while simultaneously reducing the levels of bactericidal materials and photonic energies to achieve efficacious results. The apparatus, methods of use, and methods of treatment of the present disclosure may provide sterilization after 5 to 10 seconds of simultaneous exposure to Ozone and ultraviolet (“UV”) treatments administered at levels below those that would normally be used for each treatment if administered individually. Further treatment with infrared (“IR”) may be administered simultaneous with, overlapping with, or immediately after, treatment with Ozone and UV. Such treatment with IR is similarly at levels below those that would normally be used for IR treatment administered in isolation. The simultaneous or immediately adjacent application of the sterilizing treatments has a multiplying effect that permits these lower levels for each of Ozone, UV, and IR treatment to be below the limits of safe human exposure while effectively inactivating bacteria of many varieties. The lower treatment levels of the present disclosure also advantageously allow for repeated safe use in industrial and clinical environments. Another benefit of the apparatus, methods of use, and methods of treatment of the present disclosure is overcoming use-avoidance by removing the human repulsion to smearing cold wet fluids on hands or surfaces of other objects for sterilization. Another benefit to the present disclosure is effective sterilization in a shorter period of time than traditional methods of single treatment sterilization including, for example, autoclaving objects and methods of sterilizing the surface of skin or other objects that cannot be sterilized by traditional methods (i.e., autoclaving) but which require a sterile environment. Such instances include use in medical procedures or scientific research.

In a first aspect, an apparatus is provided for sterilizing a surface of a treatment subject. The apparatus includes a housing having an opening arranged at a first end of the housing and a cavity configured to receive the treatment subject via the opening. The apparatus also includes two or more ultraviolet-generating (“UV-generating”) modules coupled to opposing interior walls of the housing. And the apparatus includes at least one gaseous Ozone generator coupled to at least one of the opposing interior walls of the at least one sterilization chamber, where the cavity of the at least one sterilization chamber is configured for delivery of Ozone to the surface of the treatment subject.

A second aspect is directed to a method for sterilizing a surface of a treatment subject. The method includes (a) inserting a treatment subject through an opening at a first end of a housing and into a cavity, (b) delivering UV from a plurality of UV-generating emitter modules positioned on opposing interior walls of the housing to the surface of the treatment subject, and (c) delivering Ozone from a plurality of gaseous Ozone generators located on the opposing interior walls of the housing to the surface of the treatment subject.

A third aspect is directed to a method of treatment that kills microbes on a surface of a treatment subject. The method includes (a) inserting a treatment subject through a first end of a housing and into a cavity defined by the housing, (b) delivering UV to the surface of the treatment subject, wherein the UV thereby inactivates or kills microbes by interrupting DNA structure of the microbes, and (c) delivering Ozone to the surface of the treatment subject, wherein the Ozone thereby inactivates or kills microbes by breaking down bacteria cell membrane lipid layers through oxidation and rupturing cell walls of the microbes.

The features, functions, and advantages that have been discussed can be achieved independently in various embodiments or may be combined in yet other embodiments, further details of which can be seen with reference to the following description and drawings.

### BRIEF DESCRIPTION OF THE DRAWINGS

Illustrative examples of various embodiments are described below in conjunction with the appended figures, wherein like reference numerals refer to like elements in the various figures, and wherein:

FIG. 1 depicts destruction of a bacteria cell 2 using UV light at the 254 nm wavelength and disrupting bacterial cell DNA 4;

FIG. 2 depicts destruction of a bacteria cell 2 through lipid layer disruption 27 by an oxidizing agent 3;

FIG. 3 depicts destruction of a bacterial cell 2 by administering UV treatment at 222 nm wavelength 5 that is absorbed by photochemical effects 1a in the bacterial cell and converted to localized heat 1b;

FIG. 4 depicts simultaneous application of Ozone 3, UV wavelengths 5 ranging between 222 nm and 254 nm, and infrared (“IR”) treatments 21 on a bacterial cell 2, penetration of the moisture layer 6 by Ozone 3 and absorption of Ozone-creating H₂O₂ 8 on top of a treatment surface 7;

FIG. 5 depicts Ozone 3 outside of the viscous hydrostatic boundary layer 10 and located above a treatment surface 7 and demonstrates increased viscosity 18 closer to the treatment surface 7;

FIG. 6 depicts the relationship of the viscosity 18 of the hydrostatic boundary layer versus the distance to the treatment surface 7 or skin 9;

FIG. 7 depicts an exploded side view of an apparatus, according to one example implementation;

FIG. 8 depicts an exploded top view of the apparatus of FIG. 7;

FIG. 9 depicts a side cross-sectional view of a treatment subject 20 disposed within a sterilization chamber of the apparatus of FIG. 7, containing UV emitters 12, an Ozone filled space 22, infrared (“IR”) emitters 21 and IR wavelengths 23 ranging from 1000 nm to 1500 nm, and Ozone vortices 22;

FIG. 10 depicts the interaction between the viscous hydrostatic boundary layers of a treatment subject 20 and UV treatment sources;

FIG. 11 depicts an infrared (“IR”) treatment source containing IR emitters 21 and IR wavelengths 23 ranging from 1000 nm to 1500 nm configured to heat a treatment surface 20 located in the space 34 arranged between UV modules 12 to thereby increase Ozone vortex velocity;

FIG. 12 depicts an Ozone treatment method, according to one example implementation, delivering gaseous Ozone 3 to a treatment surface of a negatively charged object 20 via a static-electric field 24;

FIG. 13 depicts an Ozone treatment method, according one example implementation, delivering gaseous Ozone 3 to a treatment surface via belt-driven adhesion;

FIG. 14 depicts an Ozone treatment method, according one example implementation, in which Ozone 3 is delivered under pressure via feed holes between fibers of a brush 32 to the surface of a treatment subject 7;

FIG. 15 depicts a perspective view of an apparatus, according to one example implementation;

FIG. 16 depicts a block diagram of a computing device and a computer network, according to an example implementation for use and safety;

FIG. 17A depicts a perspective view one example implementation of an apparatus;

FIG. 17B depicts a side view of the apparatus according to FIG. 17A;

FIG. 18 depicts a cross-sectional image of a treatment subject's skin showing depth of penetration of various EM wavelengths.

FIG. 19 depicts delivery of UV and IR treatments 64, according to one example implementation, via a fiber optic cable 62 having a hollow outer jacket 63 for Ozone flow 3;

FIG. 20 depicts an example implementation of an optic bundle, that includes an inner fiber optic cable 62 and a hollow outer jacket 63 configured to deliver Ozone to the treatment subject; and

FIG. 21 depicts one example implementation of sterilization of a treatment subject 20 with an optical fiber combining UV and IR delivery 64 and an Ozone generator that supplies Ozone through a separate feed duct 14.

### DETAILED DESCRIPTION

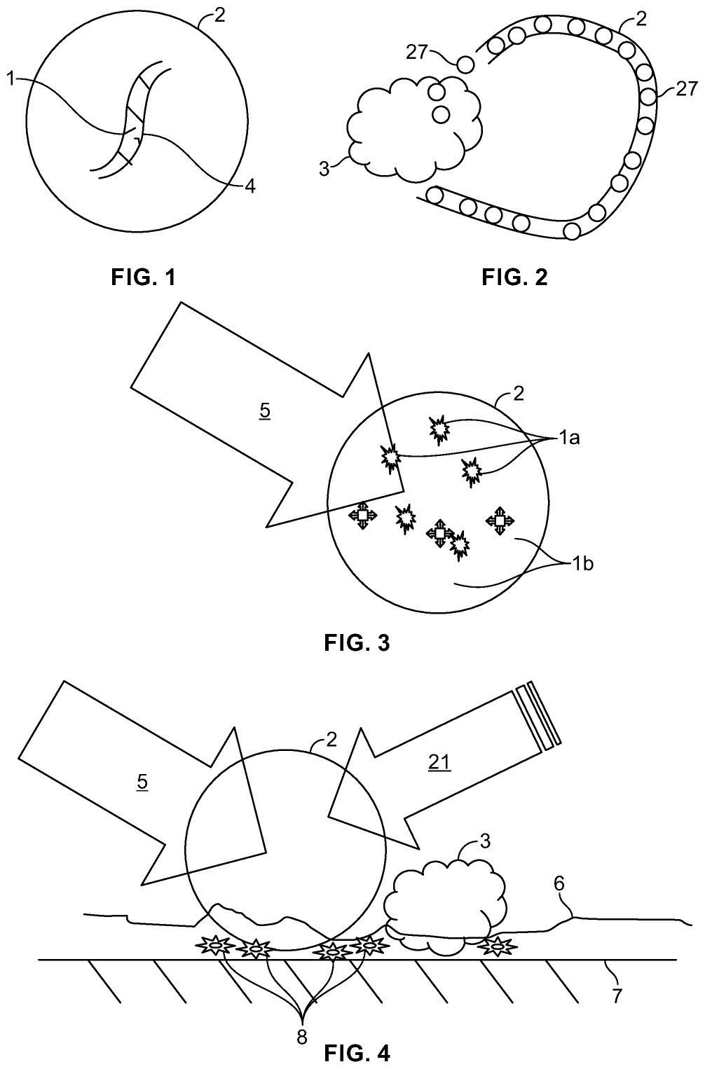

FIG. 1 illustrates a bacteria cell or bacterium 2 irradiated by UV at the 254 nm wavelength 1 to disrupt or damage DNA 4. This wavelength of UV is known to be destructive to bacteria residing on human tissues. The total energy absorption ranges from 4 mJ to 6 mJ for a given eight (8) hour period per guidelines set by the National Institute of Occupational Safety and Health (“NIOSH”). Accordingly. UV treatment must be administered under specialized and monitored conditions. The apparatus, methods of use, and methods of treatment of the present disclosure contemplate a user registering each application in a database, for example, when a human or animal is the treatment subject, and control mechanisms to avoid a user exceeding recommended dosages. The destructive modality is disruption or damage of DNA within the nucleus of the bacteria. The bacterial cell then dies as a result of DNA damage.

FIG. 2 illustrates a bacterial cell 2 being attacked by a bactericide or chemical oxidizing agent 3, including, but not limited to, chlorine, hydrogen peroxide, or Ozone. In operation, the oxidizing agent attacks the lipid layer 27 of the bacteria cell wall disrupting the cell wall integrity, resulting in bacterial cell death.

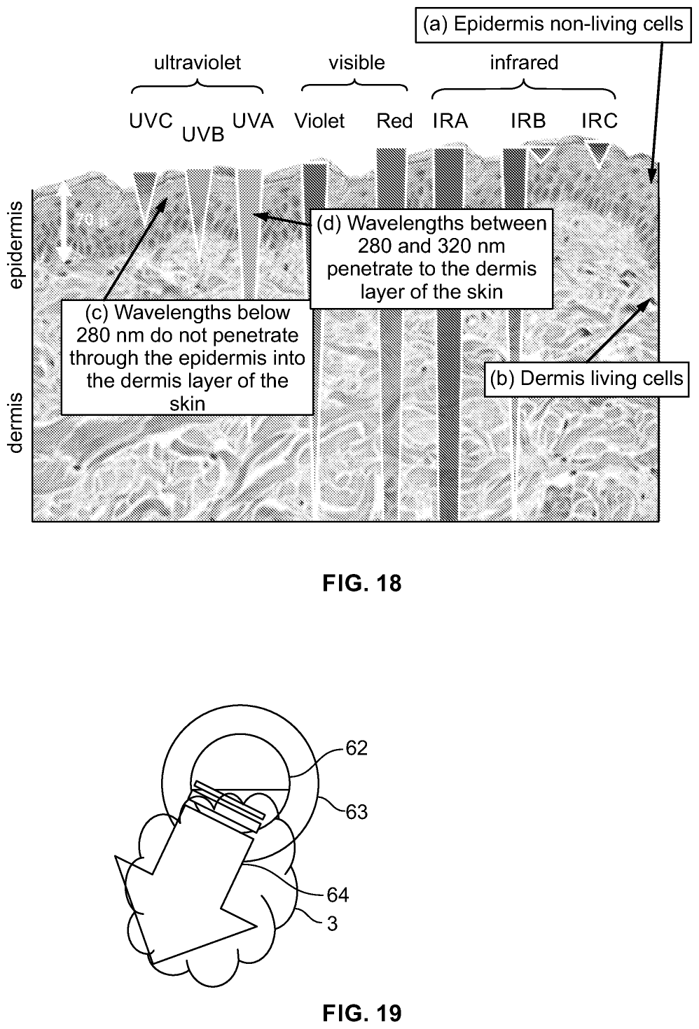

FIG. 3 shows a bacterial cell 2 exposed to UV wavelengths 5 ranging between 180 nm and 230 nm where the absorbed UV is converted to localized heat 1b thus killing the bacterial cell 2 by local mechanical heating 1b and altered bacterial cell chemistry 1a due to photochemical effects. This treatment mechanism allows for an increased bactericidal effect with a shorter wavelength, which is safer, i.e. less mutagenic, than the administration of a longer wavelength, such as a 254 nm UV wavelength. The effectiveness of 222 nm or the 254 nm wavelengths is dependent on the energy density and time, i.e. more energy or more exposure time of either wavelength results in greater bactericidal effects. The difference is in depth of penetration of each wavelength. FIG. 18 elements (b) and (d) show that UVB wavelengths penetrate 2 mm or more into the dermis. Yet UVC wavelengths do not pass to the living cell in the dermis as UVC wavelengths, as shown in element (c) of FIG. 18, including UV at a wavelength of 222 nm that are stopped by the epidermis in element (a) of FIG. 18. In various embodiments, the apparatus and methods use lower energy UV wavelengths ranging between 180 nm and 230 nm to treat the surface of skin tissue, allowing for much higher energy flux without damage to living tissue.

The present disclosure contemplates an apparatus and methods that utilize simultaneous application of photonic (i.e., UV) and chemical (i.e., Ozone) treatment, and, optionally, mechanical (i.e., infrared “IR” heat) treatment, as shown in FIG. 4. In some embodiments, UV and Ozone are applied simultaneously. In such embodiments, a multiplying effect is generated by simultaneous use of these treatments, such that if the UV treatment does not completely kill the bacteria cell 2, the weakened cell 2 may be killed via the simultaneous treatment in the form of oxidation of Ozone 3 and/or the resulting absorption of Ozone 3 into a moisture layer 8 to create hydrogen peroxide. Both oxidizing events are capable of inactivating bacteria cells. Further, should the bacteria cell 2 survive the application of both UV and Ozone treatments, application of a mild heat-source may ultimately kill the bacteria cell 2 by raising the local treatment surface temperature to 45° to 50° C. by an IR radiator 21, for example. Bacteria cells 2 in weakened state due to UV and Ozone treatments are susceptible to an increase in temperature that would be perceived by a human subject as comfortable warming. In some embodiments, the application of a mild heat-source is simultaneous with the UV and Ozone treatments. In some embodiments, the application of a mild heat-source occurs immediately after treatment with UV and Ozone.

In some embodiments, the disclosure provides use of UVA, specifically between wavelengths ranging from 320 and 400 nm, in conjunction with the UVC between 180 and 222 nm wavelengths. In such embodiments, additional wavelengths in the range from 320 to 400 nm provide a weak bactericidal effect at high energy densities and surface PH in the narrow range of 5 to 6. Wavelengths within the UVA range are known to have mutagenic effects, however heat can be delivered topically using flux levels 10 to 20 times daylight (2 to 4 mJ/cm2) or 20 to 40 mJ/cm2 at these wavelengths. The NIOSH limit for UVA wavelengths is very high with the lowest value being 7300 mJ/cm2 at 330 nm. As indicated above, usable values are well below maximum limits allowed, while affording heat and weak sterilization. UV exposure limits based on effect of UV radiation for each wavelength between 180 nm and 400 nm is available at https://orm.uottawa.ca/my-safety/em-radiation/uv/exposure-limits.

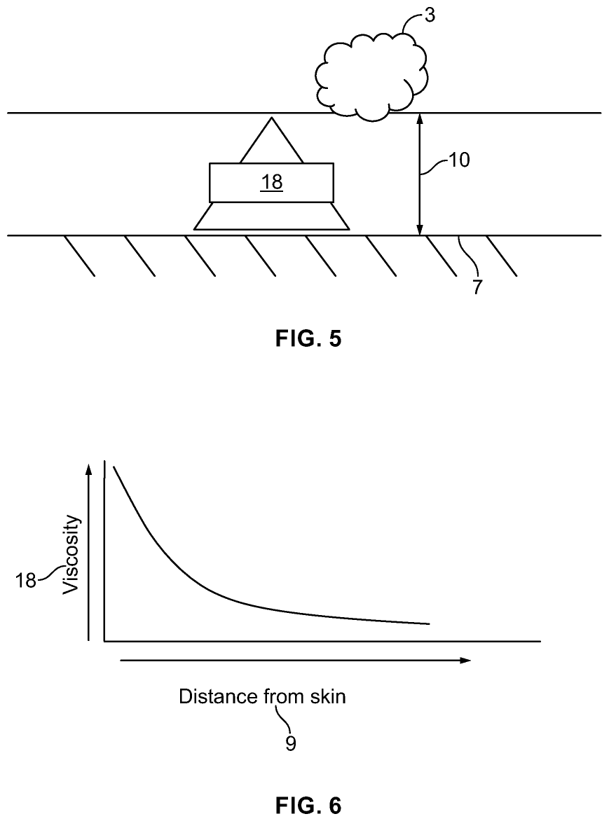

One aspect of Ozone sterilization treatment of the present disclosure is the mechanical movement of the Ozone 3 to the treatment surface 7. As seen in FIG. 5, a hydrostatic boundary layer 10 increases in viscosity 18 moving in a direction toward the treatment surface 7. The Ozone 3 permeates the region of the hydrostatic boundary layer 10 and moves about by Brownian motion (i.e., thermal molecular motion). FIG. 6 shows a graph of the relationship of viscosity 18 of the hydrostatic boundary layer versus distance 9 to the treatment surface 7. Specifically, the vertical axis corresponds to viscosity 18, and the horizontal axis reflects distance 9 to the treatment surface 7. As shown, diffusion of the Ozone slows as Ozone approaches the treatment surface 7 of FIG. 5, if Brownian motion acts alone (i.e., viscosity 18 of Ozone 3 increases as it gets closer to the treatment surface 7, such as skin).

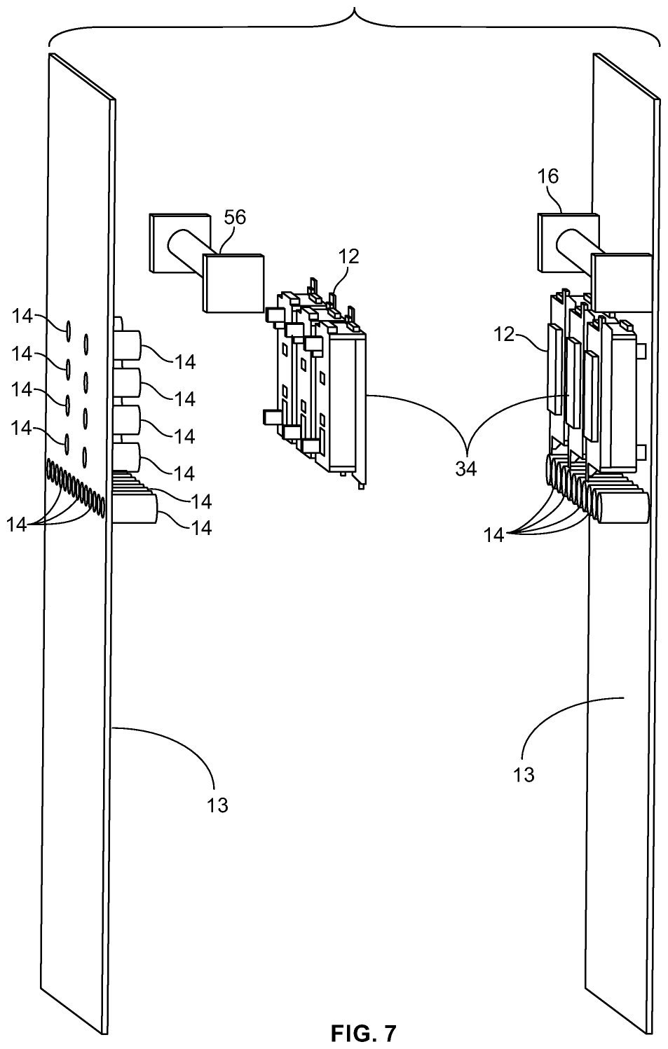

FIGS. 7-15 depict an apparatus for sterilizing a surface 7 of a treatment subject 20, that includes a housing 42, where the housing 42 has an opening 33 arranged at a first end 37 of housing 42 and has a cavity 34 configured to receive the treatment subject 20 via the opening 33. The apparatus includes two or more ultraviolet-generating (“UV-generating”) modules 12 coupled to opposing interior walls 13 of the housing 42, And the apparatus includes at least one gaseous Ozone generator 56 coupled to at least one of the opposing interior walls 13 of the housing 42, where the cavity 34 is configured for delivery of Ozone 3 to the surface 7 of the treatment subject 20.

In one optional implementation, the two or more UV generating emitter modules are configured to generate wavelengths ranging from 180 nm to 230 nm. In another optional implementation, the at least one gaseous Ozone generators 56 is configured to deliver the Ozone 3 to the surface 7 of the treatment subject 20 at or below 0.8 ppm.

In one example implementation, the apparatus further includes two or more infrared (“IR”) emitter modules 16 positioned on the opposing interior walls 16 of the housing 42. In an optional example, the two or more IR emitter modules 16 are configured to raise a local treatment surface temperature to 45° to 50° C. In FIG. 7, the IR emitter modules 16 are cylinders of quartz and the gaseous Ozone generator 56 is a cylindrical screen with a coaxial conductor in the center with HVAC, as one example.

In another implementation, the housing 42 also includes at least one blower 40 arranged in proximity to the opening 33 at the first end 37 of the housing 42 and is configured to direct the Ozone 3 back into the housing 42 such that the negative pressure permits less than 0.1 ppm of the Ozone 3 to escape the housing 42.

In one optional implementation, the apparatus is configured to sterilize the treatment subject 20 within 5 to 20 seconds of exposure to UV delivered by the UV-generating modules 12 and to the Ozone 3 delivered by the gaseous Ozone generators 56.

Methods may be employed using any of the foregoing apparatus. For example, a method for sterilizing a surface 7 of a treatment subject 20 is provided. The method includes inserting a treatment subject 20 through an opening 33 at a first end 37 of a housing 42 and into a cavity 34. Next, the method includes delivering UV from a plurality of UV-generating emitter modules 12 positioned on opposing interior walls 13 of the housing 42 to the surface 7 of the treatment subject 20. Then, the method includes delivering Ozone 3 from a plurality of gaseous Ozone generators 56 located on the opposing interior walls 13 of the housing 42 to the surface 7 of the treatment subject 20.

In a further implementation, the method further includes delivering infrared (“IR”) by two or more IR-emitter modules 16 coupled to the opposing interior walls 13 of housing 42 to the surface 7 of the treatment subject 20. In one optional example, the IR is generated and delivered simultaneously with the delivery of the UV and the delivery of the Ozone 3. In an alternative example, the IR is generated and delivered immediately after the delivery of the UV and delivery of the Ozone 3. In another implementation, delivering IR by the two or more IR-emitter modules 16 raises a local surface temperature of the treatment subject 20 to 45° to 50° C.

In one implementation, delivering the Ozone 3 from the gaseous Ozone generators 56 located on the opposing interior walls 13 of the housing 42 to the surface 7 of the treatment subject 20 further comprises at least one of: (i) generating a mechanical force 58, 59 that creates a vortex causing an interaction between hydrostatic boundary layers 10, 19 of the UV-generating emitter modules 12 and the treatment subject 20, (ii) utilizing a static-electric field, (iii) utilizing electro-deposition, (iv) disrupting a viscosity 18 between hydrostatic boundary layers of a moving belt 28 suspended between two rollers 30 disposed within the cavity 34 of the housing 42 and the surface 7 of the treatment subject 20; and (v) creating a mechanical disruption of the hydrostatic boundary layer 19 of the treatment subject 20 via a tube, a nozzle, or a duct 14 that feeds the Ozone 3 to a base of a brush 32 comprising bristles or fibers extending from the base of the brush 32.

In a further implementation, sterilizing the treatment subject 20 occurs within 5 to 20 seconds after insertion of the treatment subject 20 through the first end 37 of the housing 42 and into the cavity 34 defined by the housing 42.

The present disclosure also contemplates a method of treatment that kills microbes on a surface of a treatment subject. The method includes inserting a treatment subject 20 through a first end 37 of a housing 42 and into a cavity 34 defined by the housing 42. The method then includes delivering UV to the surface 7 of the treatment subject 20, where the UV thereby inactivates or kills microbes by interrupting DNA structure 4 of the microbes 2. Next, the method includes delivering Ozone 3 to the surface 7 of the treatment subject 20, where the Ozone 3 thereby inactivates or kills microbes by breaking down bacteria cell membrane lipid layers through oxidation and rupturing cell walls of the microbes.

In one example implementation, the method of treatment further includes delivering infrared (“IR”) to the surface 7 of the treatment subject 20. In one optional example, the IR is delivered simultaneously with delivery of the UV and delivery of the Ozone 3. In an alternative example, the IR is delivered immediately after delivery of the UV and delivery of the Ozone. In one optional implementation, delivery of the IR raises a local surface temperature of the treatment subject 20 to 45° to 50° C.

The method of treatment according to any of the foregoing implementations may further include sterilizing the treatment subject 20 within 5 to 20 seconds after insertion of the treatment subject 20 through the first end 37 of the housing 42 and into the cavity 34 defined by the housing 42.

The present disclosure contemplates several Ozone delivery methods to disrupt the hydrostatic boundary layer 10 of the treatment surface 7. In the Ozone delivery systems described herein, the intermixing of Ozone species with non-Ozone boundary species and the target pathogens can be enhanced by the application of ultrasonic energy. This may be particularly useful where the boundary of the sample to be sterilized is a hard boundary, such as metal or plastics, as the reflected wave may have twice the amplitude as a result of coincidence of the incoming and reflected wave. The enhancement is a result of the compressive wave introducing a statistical improvement in the probability of a pathogen encountering ozone species.

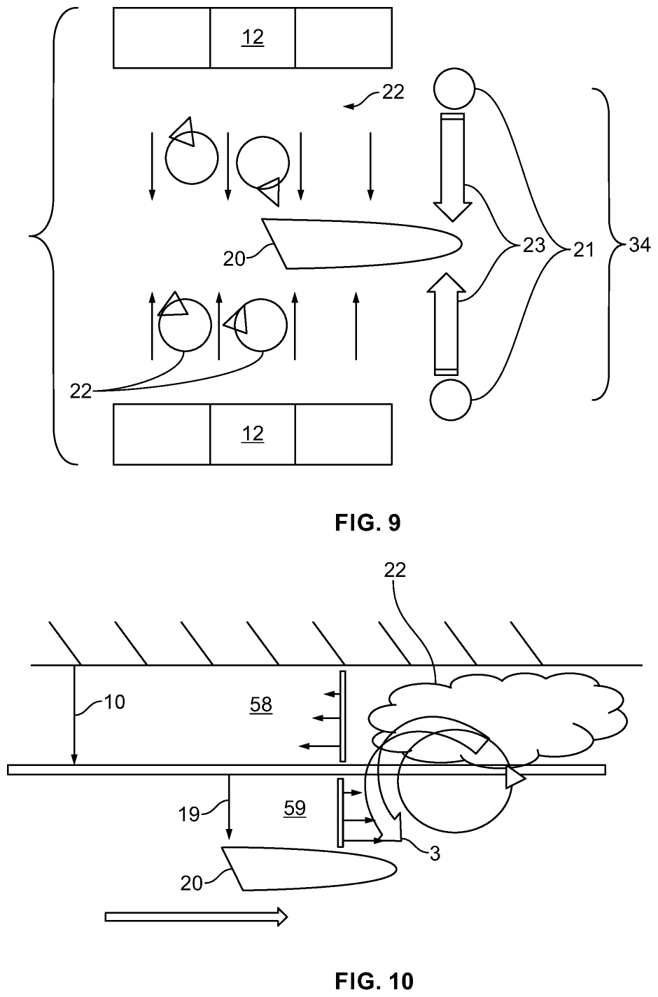

For example, in FIG. 9, a cross-section of a treatment subject 20 is shown passing between two UV-generating modules 12 with associated hydrostatic boundary layers, while the treatment subject 20 also has a hydrostatic boundary layer. FIG. 10 shows the interaction of the hydrostatic boundary layer 10 of the UV-generating modules and the hydrostatic boundary layer 19 of the treatment subject 20. The resulting mechanical forces 58, 59 at a molecular level are reflected as the reaction forces against a moving static air layer attached to the treatment subject 20. The hydrostatic boundary layer 10 is saturated with Ozone 3 in an amount up to 1.0 ppm. This Ozone-saturated hydrostatic boundary layer 10 then interacts with the hydrostatic boundary layer 19 corresponding to moving treatment subject 20. The reaction forces 58, 59 resulting from the interaction of the hydrostatic boundary layers 10, 19 create a vortex that carries the Ozone 3 to the treatment surface in a dynamic way based on the motion of the treatment subject 20 to be sterilized.

In FIG. 12, an alternative Ozone delivery method is shown. For example, a static-electric field 24 can be utilized to drive Ozone 3 to the treatment surface 7 for sterilization. The Ozone species has a net charge as a result of Ozone's polar nature. The effect is commonly known as Farad's wind. As such, once the treatment subject 20 is charged correctly with the E-field polarity aligned to accelerate the Ozone 3 to the surface, the Ozone 3 will pass through the hydrostatic boundary layer by the energy obtained due to acceleration through the sterilization space containing E-field 24. Ozone 3 can be directed to the correctly charged treatment subject to follow the polar E-field gradient direction, such that the charged O₃ molecule is accelerated to the treatment subject 20 by electro-deposition as shown in FIG. 12.

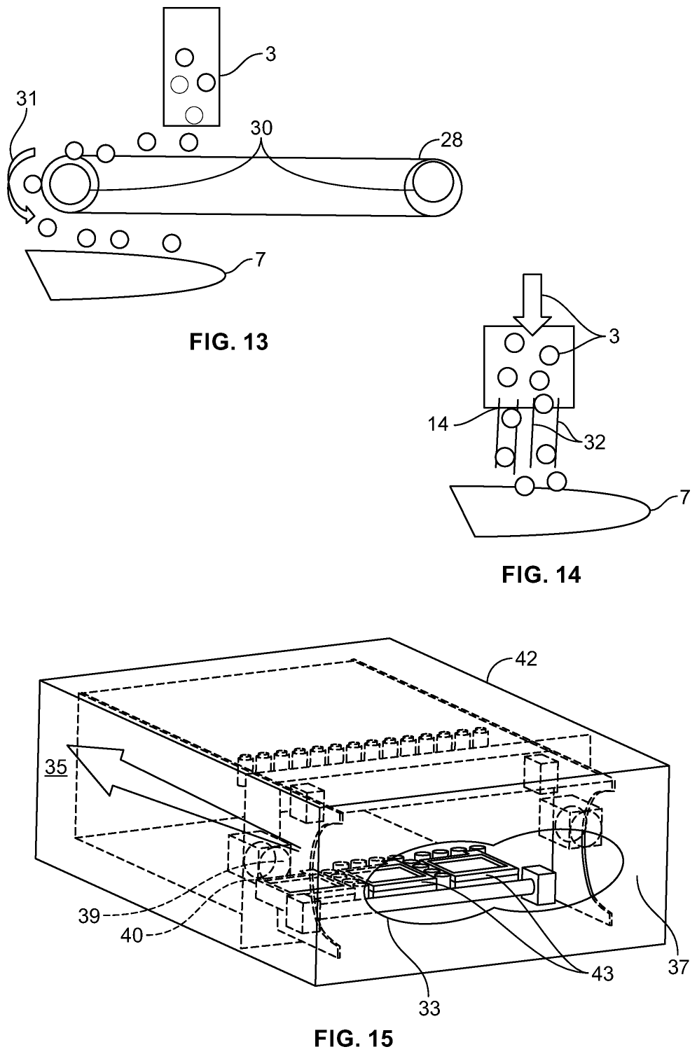

Further, Ozone, by its polar nature, is sticky and tends to cluster so as to balance the distributed E-field of the Ozone at its boundary minimum value, as described by Farad's law. This effect can be employed through a third method of Ozone delivery, as seen in FIG. 13. For example, a moving belt 28 may be suspended between two rollers 30 while Ozone 3 is brought into the proximity of the belt surface. The nature of the belt 28 could be a thick pile for the Ozone 3 to intertwine with or could be a surface that is charged to hold the Ozone by static E-field. The Ozone 3 may be delivered to the treatment surface 7 by viscosity disruption between the hydrostatic boundary layers of the treatment surface 7 and the moving belt 28, as seen in FIG. 13. This would allow, for example, the application of the Ozone 3 to a wound without direct contact with the moving belt 28. One advantage of this method is to permit Ozone deposition without creating airborne Ozone.

A fourth Ozone delivery method includes mechanical disruption of the hydrostatic boundary layer as shown in FIG. 14. Here one of the Ozone feeds ducts 14, shown in FIG. 14, and terminates in a base of fiber brush 32 having holes in the base with spaced-apart bristles or fibers coupled to and extending from the base. The spaced-apart bristles or fibers are arranged such that with the Ozone 3 flows between the fibers/bristles. As the bristles or fibers drag on the surface, Ozone species 3 is directly deposited on the surface 7 as the fiber disrupts the static air boundary layer.

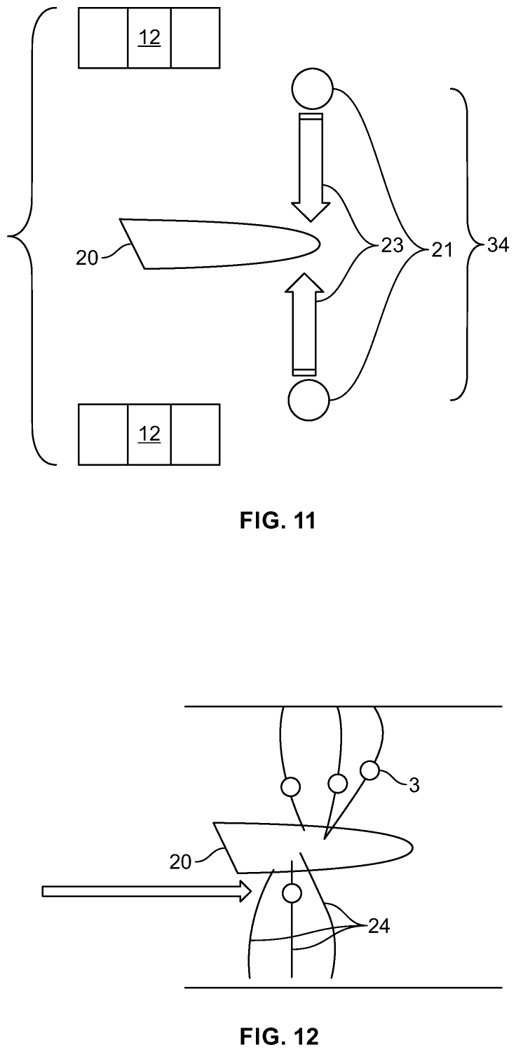

FIG. 11 shows a further sterilization method that has a multiplicity of functionality. In addition to sterilization by delivery of Ozone with UV, IR-emitters 21 deliver short-wavelength IR to further sterilize the desired treatment subject 20 in the open space 34 arranged between UV modules 12. The IR wavelength has shallow penetration that creates thermal currents 23 on the surface of the treatment subject 20 in the Ozone-saturated space 34 of the interior of the sterilization apparatus. In some embodiments, the IR is applied at the same time as, or simultaneously with, the Ozone and/or UV delivery. In some embodiments, the IR is applied after the Ozone and/or UV delivery. In some embodiments, the IR is applied immediately after the Ozone and/or UV delivery. This application method has the added benefit and technical effect of thermally stressing any remaining pathogens to increase the death rate of bacteria cells 2 located on the object to be sterilized 20.

The heating effect of the apparatus and methods of the present disclosure also beneficially create a warming sensation that has a positive psychological effect on people, thereby creating a positive feeling during use of the apparatus and methods.

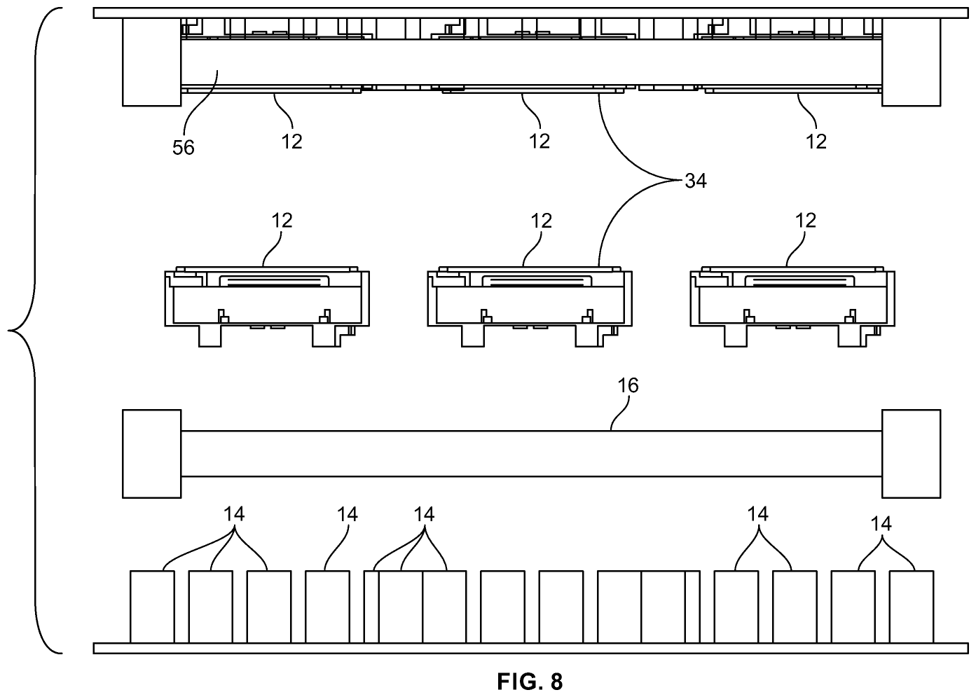

In another embodiment, the apparatus is shown in FIGS. 7 and 8, having a plurality of UV modules 12 or lamps with UV-capable focusing modules, arranged on opposing sides 13 of a sterilization chamber within a housing 42 and configured to radiate UV at a selected wavelength that ranges between 180 nm to 230 nm, for example. Feed ducts 14 are configured to deliver Ozone to the sterilization cavity 34 at a saturation level effective to weaken and/or kill a bacteria cell 2. In applications to mammalian tissue, the Ozone levels delivered to the skin tissue are at or below 0.8 ppm. The Ozone levels may be higher in the sterilization cavity 34, but the Ozone levels exterior to the apparatus must be maintained at or below a level of 0.1 ppm in any space the air may be inhaled by a user. The space 34 provided between opposing walls 13 of the sterilization chamber and therefore between the various sterilization treatment sources is contemplated to accommodate the insertion of the treatment subject 20 or object to be sterilized. The walls defining the housing 42 of the sterilization chamber are coupled to the UV, Ozone, and heat treatment sources, and these treatment sources are each configured to be operated at levels that are safe for human or animal exposure.

There are many functional arrangements of the various sterilization treatment sources within the sterilization chamber contemplated by the present disclosure. In one example, a plurality of IR-emitters or IR-radiators 16 may be uniformly distributed on two or more walls 13 within the sterilization chamber to provide a warming effect throughout the sterilization chamber. The UV module 12 may be also arranged differently than shown to better utilize the optical radiation patterns that may be generated.

UV intensity ultimately depends on the selected wavelength within the range of 180 nm to 230 nm at levels of 10 mJ/cm² and above. For a wavelength of 254 nm, NIOSH guidelines require that the UV intensity is less than 5 mJ/cm2 and require monitoring of users so that daily exposure limits are not exceeded.

In the present disclosure, multiple treatment sources are used in combination to accentuate the effects of each other, including combinations of UV and Ozone treatments, Ozone and heat treatments, and UV, Ozone, and heat treatments. Therefore, in one contemplated embodiment, the apparatus is configured to operate a minimum of two of the UV, Ozone and IR treatment sources in combination with each other and also may be sequenced in multiple steps or simultaneously delivered to increase the number of bacteria cells that are killed or weakened.

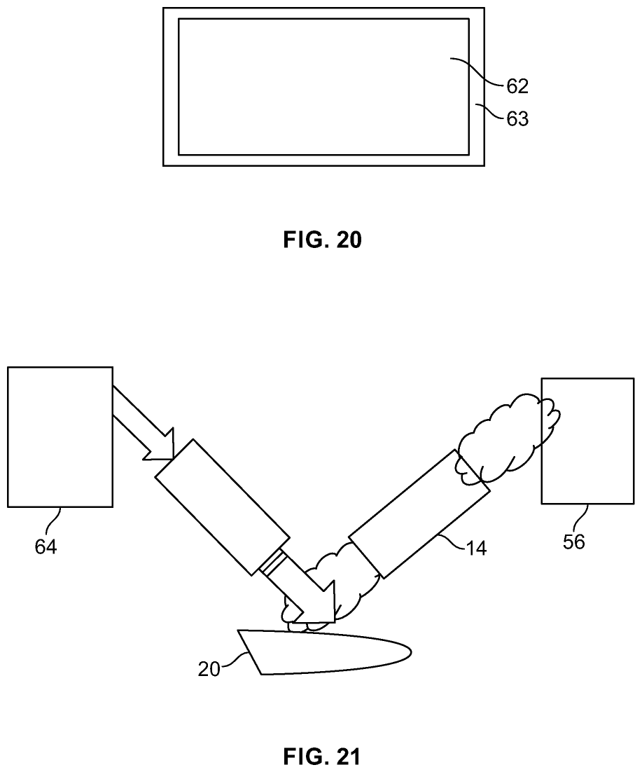

FIG. 19 shows an example method for UV and IR treatments 64 delivered together through a fiber optic cable 62, which optionally includes a hollow outer jacket 63 through which Ozone 3 may also be delivered to the surface 7 of a treatment subject 20. FIG. 20 shows an example of an optic bundle, that includes an inner fiber-optic cable 62 and a hollow outer jacket 63 through which Ozone may flow. FIG. 21 shows an exemplary treatment of a subject 20 with an optical fiber containing a combination of UV and IR 64 and a separate treatment with Ozone 3 through an Ozone generator 56 that supplies Ozone through a separate feed duct 14. In some embodiments, the UV and IR treatment is simultaneous with the Ozone treatment.

Mother view of the apparatus is provided in FIG. 15. In this embodiment the sterilization treatment sources or modules 43 are contained in a housing 42 where they are arranged to be offset from the treatment subject. The offset configuration allows for containment of the UV and Ozone. Given the optical radiation patterns, the offset distance of the UV treatment sources from the chamber opening 33 can be estimated to prevent stray UV escaping from the housing 42 of the sterilization chamber. Gaseous Ozone may be contained by air handler 40. The graduated arrow 35 shows the pressure differential between the chamber opening 33 at a proximal end that creates a closed loop air flow which prevents discharge of Ozone outside of the apparatus. A negative pressure 39 from air handlers or blowers 40 lower pressure located near the chamber opening 33 at the proximal end of the housing 42 and vacuums Ozone to prevent or minimize Ozone leaking from the housing 42 of the sterilization chamber. The blower 40 is arranged between the IR, Ozone and UV treatment modules 43 and the chamber opening 33 and directs Ozone back into the sterilization chamber. In some embodiments, there may be symmetric blowers 40 arranged on opposing sides of the sterilization chamber near the chamber opening 33 or the blower 40 may be configured as a ring arranged around the chamber opening 33. The amount of negative pressure utilized is configured to permit less than 0.1 ppm of Ozone to escape at or near the inlet opening 33 regardless of the Ozone level within the sterilization chamber.

The portion of the sterilization chamber between the proximal and distal ends of the housing 42 is open space 34 configured to receive the treatment subject or object 20. Ozone is contained within a hydrostatic boundary layer outside of the open space 34 in the sterilization chamber until the device is activated. In some embodiments, Ozone will be contained within the hydrostatic boundary layer of, for example, a belt 28, the fibers of a brush, within a nozzle or an Ozone-generating lamp. Once the device is activated, Ozone is delivered to the open space 34 in the sterilization chamber, through the hydrostatic layer around the treatment subject or object 20 and to the surface of the treatment subject or object 20 by one of several delivery mechanisms: (1) interaction of the hydrostatic boundary layers of the UV-generating modules 12 and the treatment subject or object 20 by mechanical forces that create a vortex causing an interaction of the hydrostatic boundary layers and delivery of Ozone to the surface 7 of the treatment subject or object 20 (see FIGS. 9 and 10); (2) delivery of Ozone through the hydrostatic boundary layer(s) to the surface of a treatment subject or object by a static-electric field or by electro-deposition (see FIG. 12); (3) non-airborne delivery of Ozone that is in proximity to the surface of a moving belt 28 suspended between two rollers 30 to the surface 7 of the treatment subject or object 20 by disrupting the viscosity between the hydrostatic boundary layers of the treatment surface and the moving belt 28 (see FIG. 13); (4) delivery of Ozone fed through a tube, nozzle or duct 14 (see FIGS. 7 and 8) to the base of a brush 32 with bristles or fibers extending from the base and delivered to the surface of the treatment subject or object 20 by mechanical disruption of the hydrostatic boundary layer (see FIG. 14). The open space 34 of the sterilization chamber may also be regulated at a higher air temperature for warming comfort of a treatment subject or object, as well as to kill or weaken bacteria cells. This could be accomplished by further distribution of additional IR treatment sources beyond the main sterilization chamber under the UV and Ozone treatment sources.

Ozone can also be generated with a lamp that produces 170 nm to 190 nm UV wavelengths. This will convert any free oxygen to Ozone species. Ozone-generating lamps 56 may be used as a treatment source placed within the sterilization chamber in and around the UV treatment sources, as shown in FIG. 8. These Ozone-generating lamps 56 may be powered off when the treatment subject is skin so as to limit short wave UV exposure. Alternatively, Ozone-generating lamps 56 may be placed at a distance such that Ozone 3, which both absorbs and blocks short wave UV, acts to limit exposure to the Ozone-generating lamps 56.

An alternate method of Ozone generation is coronal discharge and a corresponding Ozone generator 56 is shown in FIG. 7. In operation, a small low volume pump moves the generated Ozone from the corona device and delivers the Ozone to feed ducts 14 to be administered to the treatment subject 20 in the sterilization chamber. The distributed Ozone flow will be less than 0.5 meters per second to avoid creating turbulence that could lead to ozone leakage.

Sensors may be employed to monitor Ozone levels within the sterilization chamber and exterior to the inlet opening 33. The sensors help to maintain any Ozone leakage below 0.1 ppm and to ensure Ozone sterilization levels equal to or greater than 0.8 ppm within the sterilization chamber before a sterilization procedure begins.

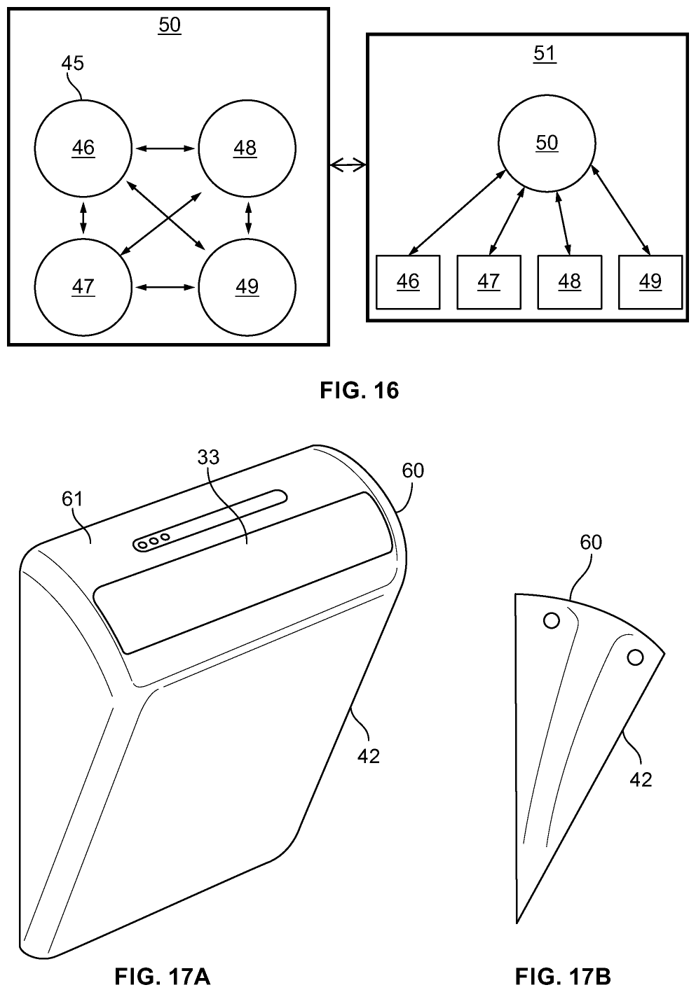

The apparatus may be configured to include a computing device 50 and a central computer network 51, according to an example implementation as shown in FIG. 16, that maintain and distribute use information corresponding to specific users so that sterilization limits and adherence to sterilization protocols are recorded and communicated to other sterilization apparatus 46-49 that are in communication with the central computer network 51 (e.g., all sterilization machines in a given hospital or laboratory environment).

User access 45 to the sterilization apparatus may be limited by ID card insertion, RFID tag, wireless access via phone, or subcutaneous implant 53, among other options, as shown in FIG. 16. The implant may also be used to provide wireless feedback to the sterilization system such as heat temperature, IR penetration, PH values and local conductivity. This data may then be relayed to an external receiver 52.

In one optional embodiment, the color of any visible light 61 emitted from the apparatus will generally be warm in color with a goal of increasing the feeling of comfort in use and to convey nonverbally that the apparatus is a positive addition to the disease fighting team. In addition, the color of any visible light 61 emitted from the apparatus would preferably not be blue, because blue light has been found by researchers to have an alerting effect, and the apparatus may be used in areas such as hospital patient rooms where patients will need to rest or sleep. likewise, the color of any visible light 61 emitted from the apparatus will preferably not be red as this color signals alarm and danger. Red may be used as a trouble annunciator that is exercised for a momentary trouble condition but should not be visible during normal operation of the apparatus.

One example configuration of the housing 42 of the apparatus is provided in FIG. 17. In this embodiment, the housing 42 has a few gentle curves 60 and contours although the housing 42 may be primarily rectilinear in form. This combination will invite use, while simultaneously signaling effectiveness and efficiency.

The foregoing detailed description is intended to be regarded as illustrative rather than limiting and the following claims, including all equivalents, are intended to define the scope of the invention. The claims should not be read as limited to the described order or elements unless stated to that effect. Therefore, all examples that come within the scope and spirit of the following claims and equivalents thereto are claimed.

1. An apparatus for sterilizing a surface of a treatment subject, the apparatus comprising:
a housing having an opening arranged at a first end of the housing and a cavity configured to receive the treatment subject via the opening;
two or more ultraviolet-generating (“UV-generating”) modules coupled to opposing interior walls of the housing; and
at least one gaseous Ozone generator coupled to at least one of the opposing interior walls of the at least one sterilization chamber, wherein the cavity of the at least one sterilization chamber is configured for delivery of Ozone to the surface of the treatment subject.

2. The apparatus according to claim 1, further comprising: two or more infrared (“IR”) emitter modules positioned on the opposing interior walls of the housing.

3. The apparatus according to claim 1, wherein the two or more UV generating emitter modules are configured to generate wavelengths ranging from 180 nm to 230 nm.

4. The apparatus according to claim 1, wherein the at least one gaseous Ozone generators is configured to deliver the Ozone to the surface of the treatment subject at or below 0.8 ppm.

5. The apparatus according to claim 1, wherein the housing further comprises at least one blower arranged in proximity to the opening at the first end of the housing and is configured to direct the Ozone back into the housing such that the negative pressure permits less than 0.1 ppm of the Ozone to escape the housing.

6. The apparatus according to claim 2, wherein the two or more IR emitter modules are configured to raise a local treatment surface temperature to 45° to 50° C.

7. The apparatus according to claim 1, wherein the apparatus is configured to sterilize the treatment subject within 5 to 20 seconds of exposure to the UV and the Ozone.

8. A method for sterilizing a surface of a treatment subject, the method comprising:
inserting a treatment subject through an opening at a first end of a housing and into a cavity;
delivering UV from a plurality of UV-generating emitter modules positioned on opposing interior walls of the housing to the surface of the treatment subject; and
delivering Ozone from a plurality of gaseous Ozone generators located on the opposing interior walls of the housing to the surface of the treatment subject.

9. The method according to claim 8, further comprising delivering infrared (“IR”) by two or more IR-emitter modules coupled to the opposing interior walls of housing to the surface of the treatment subject.

10. The method according to claim 9, wherein the IR is generated and delivered simultaneously with the delivery of the UV and the delivery of the Ozone.

11. The method according to claim 9, wherein the IR is generated and delivered immediately after the delivery of the UV and delivery of the Ozone.

12. The method according to claim 9, wherein delivering IR by the two or more IR-emitter modules raises a local surface temperature of the treatment subject to 45° to 50° C.

13. The method according to claim 8, wherein delivering the Ozone from the gaseous Ozone generators located on the opposing interior walls of the housing to the surface of the treatment subject further comprises at least one of:
(i) generating a mechanical force that creates a vortex causing an interaction between hydrostatic boundary layers of the UV-generating emitter modules and the treatment subject;
(ii) utilizing a static-electric field;
(iii) utilizing electro-deposition;
(iv) disrupting a viscosity between hydrostatic boundary layers of a moving belt suspended between two rollers disposed within the cavity of the housing and the surface of the treatment subject; and
(v) creating a mechanical disruption of the hydrostatic boundary layer of the treatment subject via a tube, a nozzle, or a duct that feeds the Ozone to a base of a brush comprising bristles or fibers extending from the base of the brush.

14. The method according to claim 8, further comprising:
sterilizing the treatment subject occurs within 5 to 20 seconds after insertion of the treatment subject through the first end of the housing and into the cavity defined by the housing.

15. A method of treatment that kills microbes on a surface of a treatment subject, the method comprising:
inserting a treatment subject through a first end of a housing and into a cavity defined by the housing;
delivering UV to the surface of the treatment subject, wherein the UV thereby inactivates or kills microbes by interrupting DNA structure of the microbes; and
delivering Ozone to the surface of the treatment subject, wherein the Ozone thereby inactivates or kills microbes by breaking down bacteria cell membrane lipid layers through oxidation and rupturing cell walls of the microbes.

16. The method according to claim 15, further comprising: delivering infrared (“IR”) to the surface of the treatment subject.

17. The method according to claim 16, wherein the IR is delivered simultaneously with delivery of the UV and delivery of the Ozone.

18. The method according to claim 16, wherein the IR is delivered immediately after delivery of the UV and delivery of the Ozone.

19. The method according to claim 16, wherein delivery of the IR raises a local surface temperature of the treatment subject to 45° to 50° C.

20. The method of treatment according to claim 15, further comprising:
sterilizing the treatment subject within 5 to 20 seconds after insertion of the treatment subject through the first end of the housing and into the cavity defined by the housing.
